(19) United States
(12) Patent Application Publication
(10) Pub. No.: US 2026/0114842 A1
(43) Pub. Date: Apr. 30, 2026

(54) ENGINEERING AND IMAGING OF ECHOGENIC PHAGOCYTOTIC CELLS

(71) Applicant: Georgia Tech Research Corporation, Atlanta, GA (US)

(72) Inventors: Costas Arvanitis, Atlanta, GA (US); Ashley Alva, Atlanta, GA (US)

(21) Appl. No.: 18/854,293

(22) PCT Filed: Apr. 7, 2023
(86) PCT No.: PCT/US2023/065524

§ 371 Date: Oct. 4, 2024

Related U.S. Application Data
(60) Provisional application No. 63/362,632, filed on Apr. 7, 2022.

(51) Int. Cl. A61B 8/08; A61K 9/107; A61K 35/15; A61K 49/22; C12N 5/0786

(52) U.S. Cl. CPC A61B 8/085; A61K 9/107; A61K 35/15; A61K 49/223; C12N 5/0645

(57) ABSTRACT

An exemplary embodiment of the present disclosure provides a method for diagnostic imaging including, labeling a phagocytotic cell with an echogenic microbubble, introducing the labeled phagocytotic cell into a patient, and ultrasound imaging the patient to determine a location of the labeled phagocytotic cell in the patient.

### CROSS-REFERENCE TO RELATED APPLICATIONS

This application claims the benefit of U.S. Provisional Application Ser. No. 62/362,632, filed on 7 Apr. 2022, which is incorporated herein by reference in its entirety as if fully set forth below.

### FIELD OF THE DISCLOSURE

The various embodiments of the present disclosure relate generally to medical imaging and treatment, and more particularly to ultrasound imaging using contrast agents.

### BACKGROUND

Because of their role in the resolution and progression of many diseases, imaging of macrophage, dendritic cell, and antigen presenting cell trafficking can reveal important molecular, cellular, and functional characteristics of the host tissue and support the discovery of new biomarkers for improved diagnosis, prognosis, and treatment monitoring of human disease. While recent advances in immune cell labeling and imaging have provided new capabilities in imaging macrophage trafficking, imaging methods with high sensitivity that are also able to effectively track individual macrophages deep in the body as they interact with the host and over long timescales remains an unmet need.

While macrophage and other cell imaging methods using optical microscopy, magnetic resonance imaging (MRI), computed tomography (CT), and positron emission tomography (PET) scans, are well developed, these modalities offer suboptimal tradeoffs between depth of penetration, specificity, sensitivity, and resolution. MRI has been widely used for tracking cells particularly with magnetite (Fe2O3)-based superparamagnetic iron oxide nanoparticles. However, while this method is sensitive (106 cells per ml) and has whole body imaging capability, it does lack specificity. Fluorine-19 (19F) MRI addresses the specificity issue with iron-based cell tracking as endogenous 19F is low but suffers in terms of sensitivity. PET provides whole body imaging capability and excellent sensitivity (106 cells per ml) but is limited in terms of spatial resolution. On the other hand, while optical imaging and CT have had successful preclinical trials, they face several challenges in scaling up. Optical imaging provides single cell resolution but has limited depth penetration. CT has whole body imaging capability but has low sensitivity and requires high doses of radiopaque agents to accumulate. Thus, complementary cell tracking methods are needed to support the discovery and clinical translation of macrophage-based diagnostics and therapeutic interventions. High sensitivity, specificity, and penetration depth without compromising resolution while also providing portability and potentially lower costs are requirements for this imaging technology.

Although ultrasound (US) could potentially provide a viable solution towards addressing these challenges, there is a paucity of options for using this imaging modality to image cells in general and macrophages in particular. This is due to the inherently low compressibility and density differences of the macrophages with the host cells that makes their scattering cross-section essentially identical, and as a result they produce poor image contrast.

### BRIEF SUMMARY

The present disclosure relates to methods for diagnostic imaging. An exemplary embodiment of the present disclosure provides a method for diagnostic imaging including, labeling a phagocytotic cell with an echogenic microbubble, introducing the labeled phagocytotic cell into a patient, and ultrasound imaging the patient to determine a location of the labeled phagocytotic cell in the patient.

In any of the embodiments disclosed herein, the method can further include isolating the phagocytotic cell from the patient.

In any of the embodiments disclosed herein, labeling the phagocytotic cell can include placing the phagocytotic cell in a solution comprising the echogenic microbubble, the solution contained in a sealable container, fixing the phagocytotic cells to a bottom surface of the sealable container, sealing the sealable container, inverting the sealable container, incubating the phagocytotic cell for a period of time, and washing the phagocytotic cell.

In any of the embodiments disclosed herein, the method can further include characterizing a trafficking pattern of the labeled phagocytotic cell.

Another embodiment of the present disclosure provides a method of treating a disease. The method can include performing cell engineering on a phagocytotic cell, labeling the phagocytotic cell with an echogenic microbubble, introducing the labeled phagocytotic cell into a patient, and ultrasound imaging the patient to determine a location of the labeled phagocytotic cell in the patient.

In any of the embodiments disclosed herein, the method can further include isolating the phagocytotic cell from the patient.

In any of the embodiments disclosed herein, labeling the phagocytotic cell can include placing the phagocytotic cell in a solution comprising the echogenic microbubble, the solution contained in a sealable container, fixing the phagocytotic cells to a bottom surface of the sealable container, sealing the sealable container, inverting the sealable container, incubating the phagocytotic cell for a period of time, and washing the phagocytotic cell.

In any of the embodiments disclosed herein, the period of time can be approximately 4 hours.

In any of the embodiments disclosed herein, incubating the phagocytotic cell can include incubating the cell at approximately 100 degrees Fahrenheit.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of between approximately 1:1 and 1:50.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:1.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:5.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:10.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:20.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:50.

In any of the embodiments disclosed herein, wherein the phagocytotic cell can include a macrophage.

In any of the embodiments disclosed herein, the phagocytotic cell can include a bone marrow-derived macrophage.

In any of the embodiments disclosed herein, the echogenic microbubble can include a fluorocarbon compound in a lipid shell.

In any of the embodiments disclosed herein, the fluorocarbon compound can include one or more of octoflouropropane and perflourobutane.

In any of the embodiments disclosed herein, the echogenic microbubble can include a fluorocarbon compound in a mannose shell.

In any of the embodiments disclosed herein, the fluorocarbon compound can include one or more of octoflouropropane and perflourobutane.

In any of the embodiments disclosed herein, the method can further include characterizing a trafficking pattern of the labeled phagocytotic cell.

In any of the embodiments disclosed herein, characterizing the trafficking pattern can include determining a velocity of the labeled phagocytotic cell. A low velocity of the labeled phagocytotic cell correlates with a strength of an interaction of the phagocytotic cell with a target anatomy. In any of the embodiments disclosed herein, characterizing the trafficking pattern can include determining accumulation of the plurality of phagocytotic cells. In any of the embodiments disclosed herein, characterizing the trafficking pattern can include determining a flux of the plurality of phagocytotic cells. In any of the embodiments disclosed herein, characterizing the trafficking pattern can include determining a distribution of the plurality of phagocytotic cells.

In any of the embodiments disclosed herein, performing cell engineering can include changing a phenotype of the phagocytotic cell.

In any of the embodiments disclosed herein, the method can further include determining a target site based on the trafficking pattern of the phagocytotic cell and administering a therapeutic to the target site.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a flowchart for a method of diagnostic imaging, in accordance with an exemplary embodiment of the present invention.

FIG. 1B provides a flowchart for a method of diagnostic imaging, in accordance with an exemplary embodiment of the present invention.

FIG. 2 provides a flowchart for a method of diagnostic imaging, in accordance with an exemplary embodiment of the present invention.

FIG. 3A provides a flowchart for a method of treating disease, in accordance with an exemplary embodiment of the present invention.

FIG. 3B provides a flowchart for a method of treating disease, in accordance with an exemplary embodiment of the present invention.

FIG. 4 provides a flowchart for a method of treating disease, in accordance with an exemplary embodiment of the present invention.

FIG. 5 provides a flowchart for a method of treating disease, in accordance with an exemplary embodiment of the present invention.

FIG. 6 provides an illustration of example components used in the aforementioned methods, in accordance with an exemplary embodiment of the present invention.

FIG. 7A provides illustrations of a plurality echogenic microbubbles, in accordance with an exemplary embodiment of the present invention.

FIG. 7B provides an illustration of method steps for labeling a phagocytotic cell, in accordance with an exemplary embodiment of the present invention.

FIG. 7C provides images of a phagocytotic cell, in accordance with an exemplary embodiment of the present invention.

FIG. 8A-8F provide images of a phagocytotic cell, in accordance with an exemplary embodiment of the present invention.

FIG. 9A provides a plot showing uptake of various types of echogenic microbubbles by phagocytotic cells, in accordance with an exemplary embodiment of the present invention.

FIG. 9B provides a plot showing uptake of echogenic microbubbles by phagocytotic cells over time, in accordance with an exemplary embodiment of the present invention.

FIG. 9C provides a plot showing uptake of echogenic microbubbles by phagocytotic cells over time, in accordance with an exemplary embodiment of the present invention.

FIG. 10A-10F provides images of phagocytotic cells, in accordance with an exemplary embodiment of the present invention.

FIG. 11A provides a plot of Contrast-to-Noise-Ratio, in accordance with an exemplary embodiment of the present invention.

FIG. 11B provides a schematic indicating that inertial cavitation of phagocytosed bubbles will lead to cell death, in accordance with an exemplary embodiment of the present invention.

FIG. 11C provides an experimental setup used to characterize acoustic response and cell viability of labeled macrophages, in accordance with an exemplary embodiment of the present invention.

FIG. 11D provides normalized power spectrum of the recorded acoustic emissions of unlabeled macrophages following ultrasonic excitation using the setup of FIG. 11C, in accordance with an exemplary embodiment of the present invention.

FIG. 11E provides normalized power spectrum of the recorded acoustic emissions of labeled macrophages following ultrasonic excitation using the setup of FIG. 11C, in accordance with an exemplary embodiment of the present invention.

FIG. 12A provides an image of macrophages before ultrasound imaging, in accordance with an exemplary embodiment of the present invention.

FIG. 12B provides an image of macrophages after ultrasound imaging, in accordance with an exemplary embodiment of the present invention.

FIG. 12C provides an image of labeled macrophages before ultrasound imaging, in accordance with an exemplary embodiment of the present invention.

FIG. 12D provides an image of labeled macrophages after ultrasound imaging, in accordance with an exemplary embodiment of the present invention.

FIG. 13A provides a plot showing cell survival before and after acoustic exposure, in accordance with an exemplary embodiment of the present invention.

FIG. 13B provides a plot showing cell survival before and after acoustic exposure with emphasis on wideband (WB) exposure and lack thereof, in accordance with an exemplary embodiment of the present invention.

FIG. 14A provides an ultrasound image of micro-vessels, in accordance with an exemplary embodiment of the present invention.

FIG. 14B provides an image showing the maximum intensity projection of the contrast enhanced ultrasound image stack of the vessels of FIG. 14A, in accordance with an exemplary embodiment of the present invention.

FIG. 14C provides optical microscopy images of the vessels of FIG. 14A, in accordance with an exemplary embodiment of the present invention.

FIG. 15A-15B provide normalized profiles at the labeled locations in FIG. 14A, in accordance with an exemplary embodiment of the present invention.

FIG. 16A provides images of macrophages, in accordance with an exemplary embodiment of the present invention.

FIG. 16B provides images of microbubbles, in accordance with an exemplary embodiment of the present invention.

FIG. 16C provides images of labeled macrophages, in accordance with an exemplary embodiment of the present invention.

FIG. 16D provides a plot of Contrast-to-Noise-Ratio, in accordance with an exemplary embodiment of the present invention.

FIG. 16E provides images of labeled macrophages in a target anatomy, in accordance with an exemplary embodiment of the present invention.

FIG. 17A provides an image of macrophage accumulation in a target anatomy, in accordance with an exemplary embodiment of the present invention.

FIG. 17B provides a magnified view of FIG. 17A, in accordance with an exemplary embodiment of the present invention.

FIG. 17C provides a magnified view of FIG. 17B, in accordance with an exemplary embodiment of the present invention.

FIG. 18 provides a schematic of an ultrasound framework, in accordance with an exemplary embodiment of the present invention.

FIG. 19 provides an illustration of example components used in the aforementioned methods, in accordance with an exemplary embodiment of the present invention.

FIG. 20A provides images of labeled macrophages, in accordance with an exemplary embodiment of the present invention.

FIG. 20B provides a plot showing macrophages remaining labeled over time, in accordance with an exemplary embodiment of the present invention.

FIG. 20C provides a plot showing contrast over background results for labeled and unlabeled macrophages before and after reintroduction into a patient, in accordance with an exemplary embodiment of the present invention.

FIG. 20D provides images of labeled macrophages, in accordance with an exemplary embodiment of the present invention.

FIG. 20E provides images of labeled macrophages, in accordance with an exemplary embodiment of the present invention.

FIG. 20F provides images of labeled macrophages, in accordance with an exemplary embodiment of the present invention.

FIG. 21A provides images in an experimental workflow and results of said experimental workflow, in accordance with an exemplary embodiment of the present invention.

FIG. 21B provides images of a target anatomy in the experimental workflow of FIG. 21A.

FIG. 22 provides an experimental workflow for verification of macrophage labeling, in accordance with an exemplary embodiment of the present invention.

FIG. 23 provides a schematic and images of macrophage delabeling or lack thereof over time, in accordance with an exemplary embodiment of the present invention.

FIG. 24A provides a schematic of an experimental workflow, in accordance with an exemplary embodiment of the present invention.

FIG. 24B provides images of macrophages in the experiment shown in FIG. 24A.

FIG. 24C provides a plot showing results of the experiment shown in FIG. 24A.

FIG. 25A provides a schematic of an experimental workflow, in accordance with an exemplary embodiment of the present invention.

FIG. 25B provides a plot showing results of the experiment shown in FIG. 25A, in accordance with an exemplary embodiment of the present invention.

FIG. 25C provides images of macrophages in the experiment shown in FIG. 25A.

FIG. 25D provides a plot showing results of the experiment shown in FIG. 25A, in accordance with an exemplary embodiment of the present invention.

FIG. 25E provides images of macrophages in the experiment shown in FIG. 25A.

FIG. 25F provides images of macrophages in the experiment shown in FIG. 25A.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

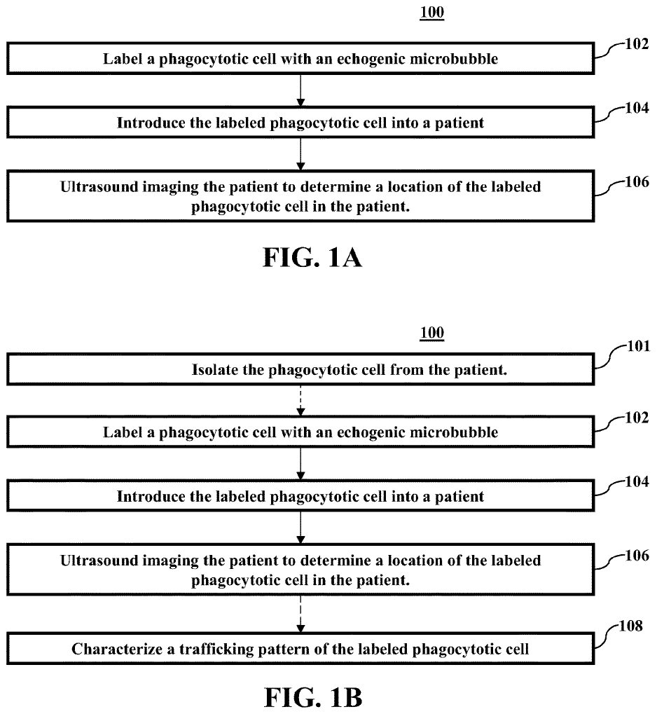

The present disclosure relates to methods for diagnostic imaging. An exemplary embodiment of the present disclosure is shown in FIG. 1A, which provides a method (100) for diagnostic imaging including, labeling (102) a phagocytotic cell with an echogenic microbubble, introducing (104) the labeled phagocytotic cell into a patient, and ultrasound imaging (106) the patient to determine a location of the labeled phagocytotic cell in the patient.

As shown in FIG. 1B, in any of the embodiments disclosed herein, the method can further include isolating (201) the phagocytotic cell from the patient.

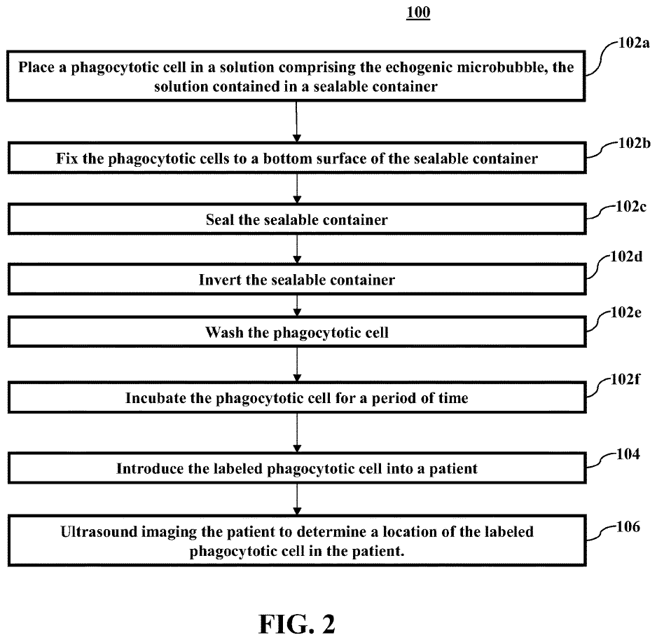

As shown in the method flowchart of FIG. 2, labeling (102) the phagocytotic cell can include placing (102a) the phagocytotic cell in a solution comprising the echogenic microbubble, the solution contained in a sealable container, fixing (102b) the phagocytotic cells to a bottom surface of the sealable container, sealing (102c) the sealable container, inverting the sealable container, incubating (102f) the phagocytotic cell for a period of time, and washing the phagocytotic cell.

In any of the embodiments disclosed herein, the period of time can be approximately 4 hours.

In any of the embodiments disclosed herein, incubating (102f) the phagocytotic cell can include incubating (102f) the cell at approximately 100 degrees Fahrenheit.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of between approximately 1:1 and 1:50.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:1.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:5.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:10.

In any of the embodiments disclosed herein, wherein the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:20.

In any of the embodiments disclosed herein, wherein the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:50.

In any of the embodiments disclosed herein, the method (100) can further include characterizing (108) a trafficking pattern of the labeled phagocytotic cell.

In any of the embodiments disclosed herein, characterizing (108) the trafficking pattern can include determining a velocity of the labeled phagocytotic cell, and the velocity of the labeled phagocytotic cell correlates with a strength of an interaction of the phagocytotic cell with a target anatomy. In any of the embodiments disclosed herein, characterizing the trafficking pattern can include determining accumulation of the plurality of phagocytotic cells. In any of the embodiments disclosed herein, characterizing the trafficking pattern can include determining a flux of the plurality of phagocytotic cells. In any of the embodiments disclosed herein, characterizing the trafficking pattern can include determining a distribution of the plurality of phagocytotic cells.

In any of the embodiments disclosed herein, wherein the phagocytotic cell can include a macrophage.

In any of the embodiments disclosed herein, the phagocytotic cell can include a bone marrow-derived macrophage.

In any of the embodiments disclosed herein, the fluorocarbon compound can include one or more of octoflouropropane and perflourobutane.

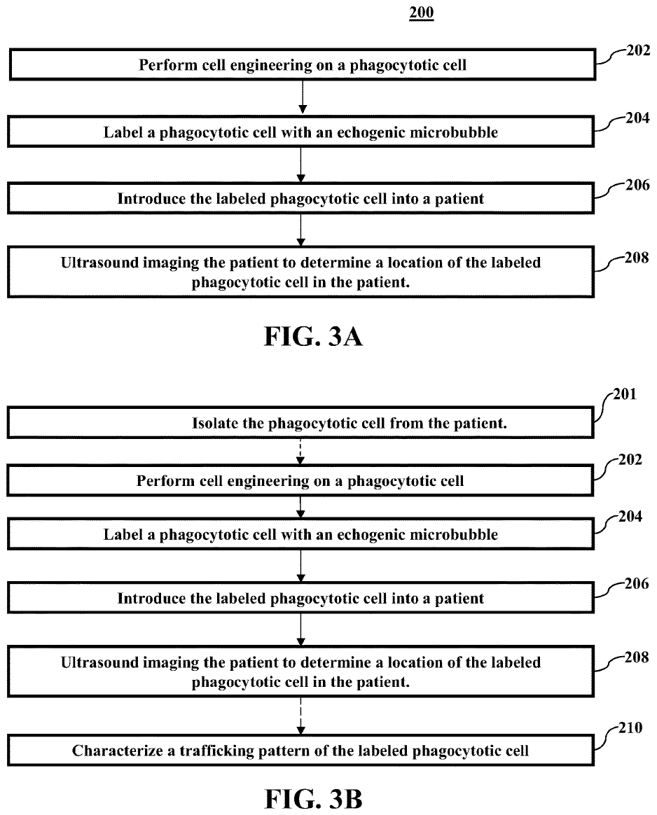

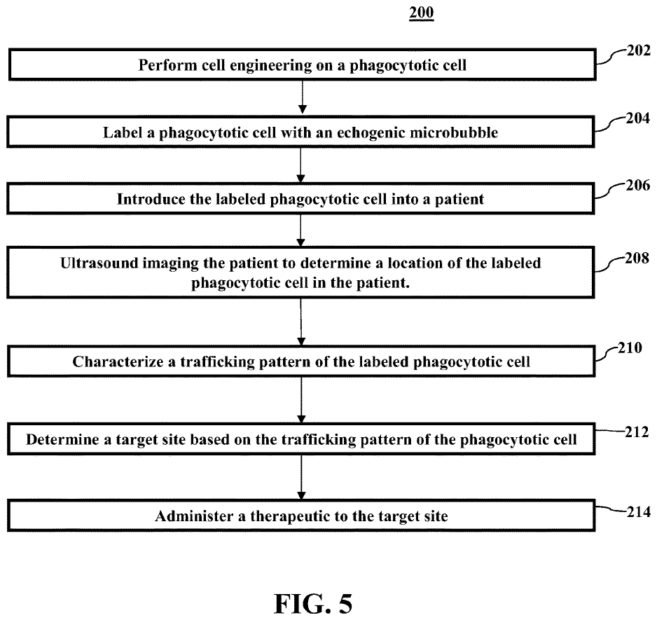

FIGS. 3A-5 show another embodiment of the present disclosure, providing a method (200) of treating a disease. The method (200) can include performing (202) cell engineering on a phagocytotic cell, labeling (204) the phagocytotic cell with an echogenic microbubble, introducing (206) the labeled phagocytotic cell into a patient, and ultrasound imaging (208) the patient to determine a location of the labeled phagocytotic cell in the patient.

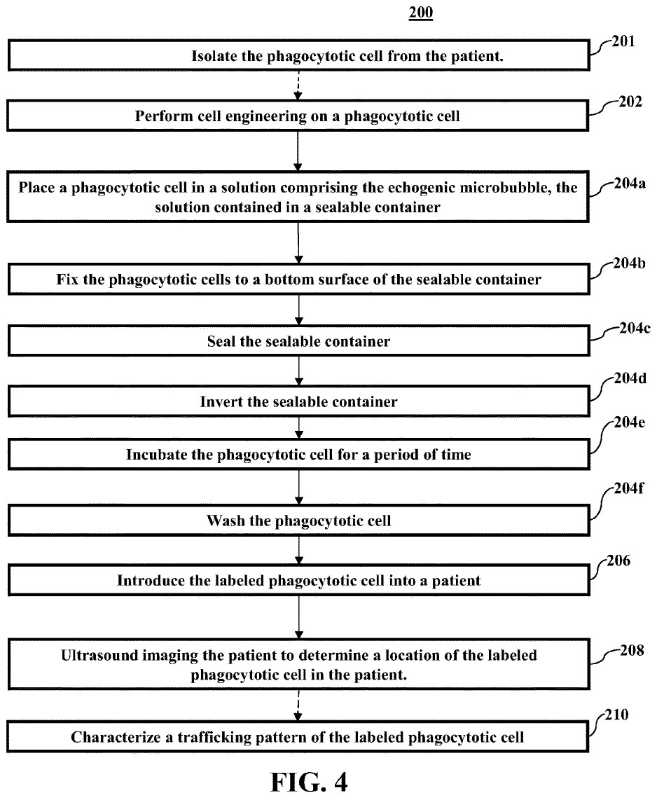

As shown in the embodiment of FIG. 4, labeling (204) the phagocytotic cell can include placing (204a) the phagocytotic cell in a solution comprising the echogenic microbubble, the solution contained in a sealable container, fixing (204b) the phagocytotic cells to a bottom surface of the sealable container, sealing (204c) the sealable container, inverting (204d) the sealable container, incubating (204e) the phagocytotic cell for a period of time, and washing (204f) the phagocytotic cell.

In any of the embodiments disclosed herein, the period of time can be approximately 4 hours.

In any of the embodiments disclosed herein, incubating the phagocytotic cell can include incubating the cell at approximately 100 degrees Fahrenheit.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of between approximately 1:1 and 1:50.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:1.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:5.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:10.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:20.

In any of the embodiments disclosed herein, the echogenic microbubble can be one of a plurality of echogenic microbubbles, wherein the phagocytotic cell can be one of a plurality of phagocytotic cells, and wherein the solution includes a ratio of phagocytotic cells to echogenic microbubbles of approximately 1:50.

In any of the embodiments disclosed herein, the method can further include characterizing (210) a trafficking pattern of the labeled phagocytotic cell.

In any of the embodiments disclosed herein, characterizing (210) the trafficking pattern can include determining a velocity of the labeled phagocytotic cell, and the velocity of the labeled phagocytotic cell correlates with a strength of an interaction of the phagocytotic cell with a target anatomy.

In any of the embodiments disclosed herein, performing (202) cell engineering can include changing a phenotype of the phagocytotic cell.

In any of the embodiments disclosed herein, the method can further include determining a target site based on the trafficking pattern of the phagocytotic cell and administering a therapeutic to the target site.

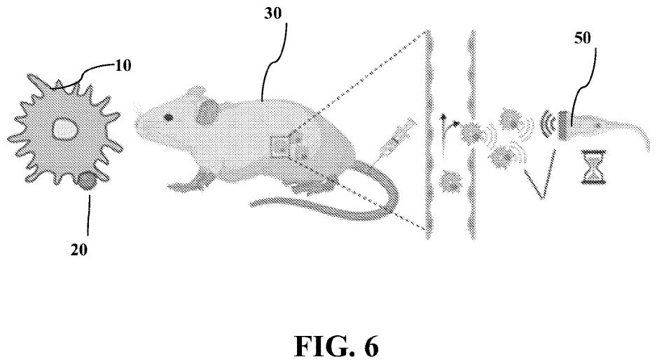

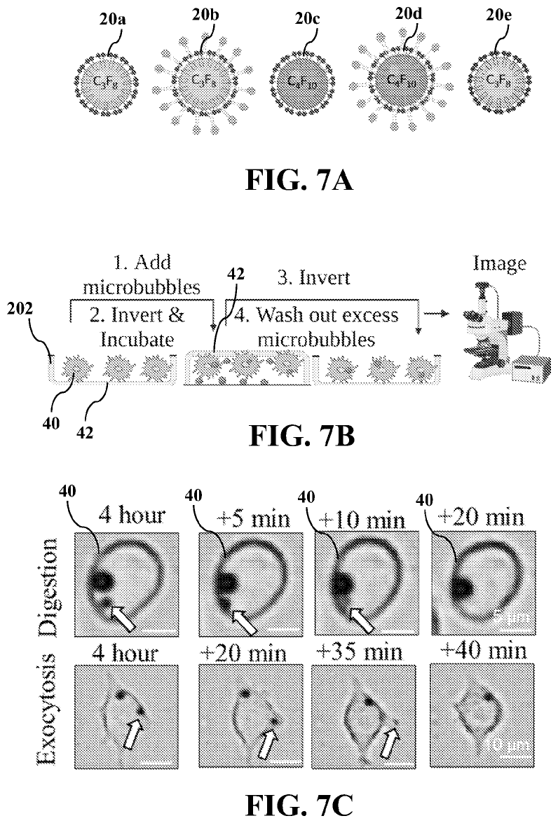

Any of the methods described herein can be employed using components shown in the figures, for example, FIG. 6-7B, such as phagocytotic cell (10), echogenic microbubble (20), patient (30), and sealable container (40) having a bottom surface (42), and ultrasound device (50) or using other components as would be understood by those skilled in the pertinent art upon reading this disclosure.

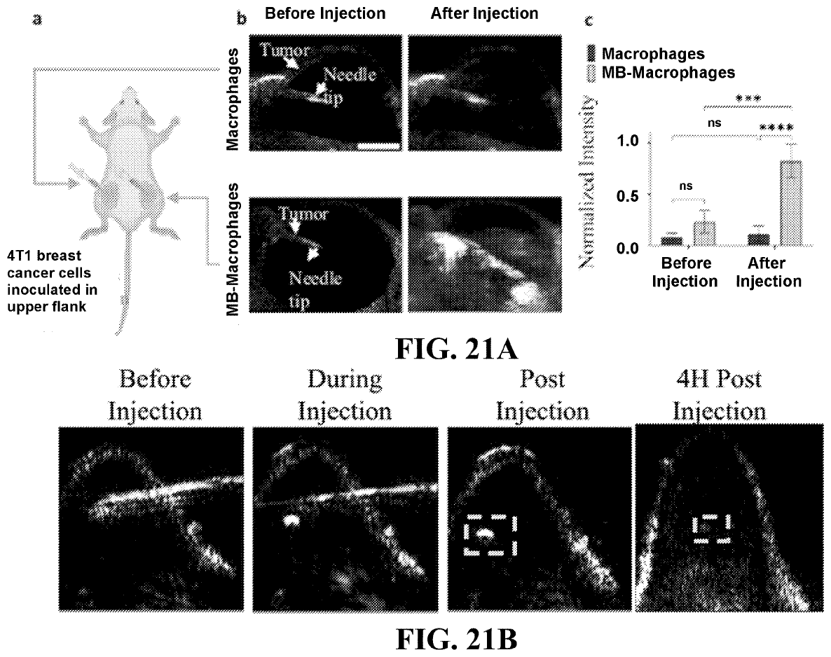

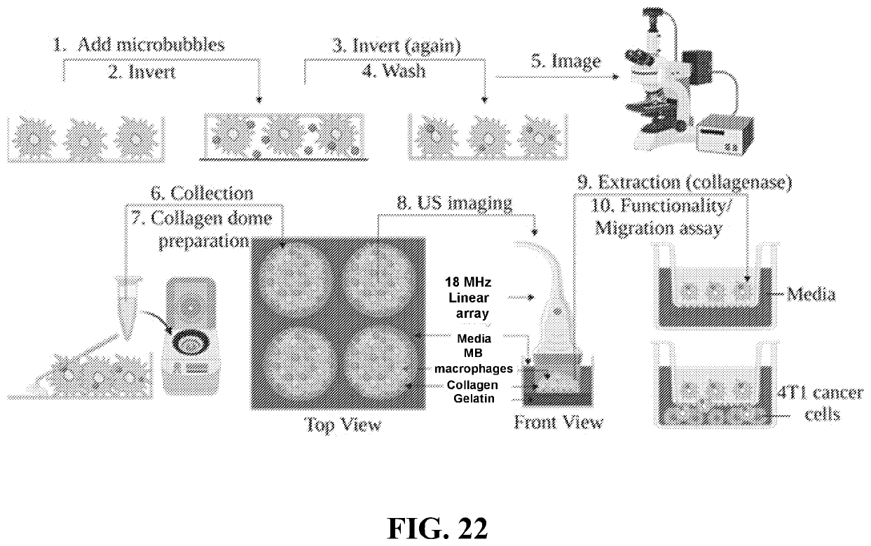

FIG. 21A provides images in an experimental workflow and results of said experimental workflow, in accordance with an exemplary embodiment of the present invention. FIG. 21B provides images of a target anatomy in the experimental workflow of FIG. 21A, described in more detail below.

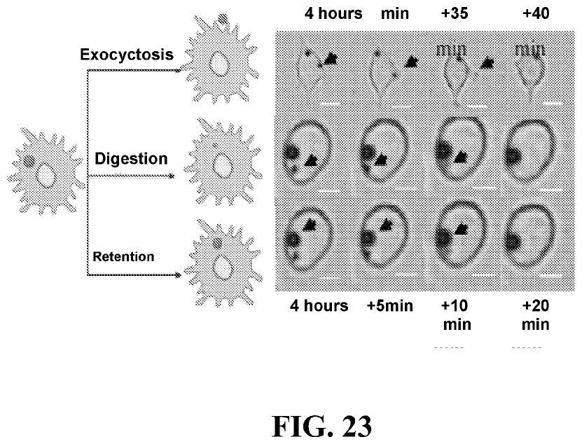

FIG. 23 provides a schematic and images of macrophage delabeling or lack thereof over time, in accordance with an exemplary embodiment of the present invention.

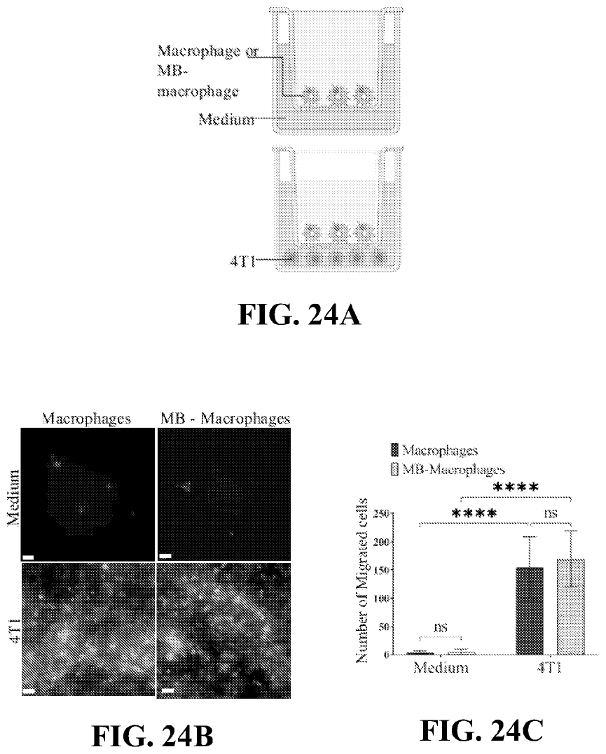

FIG. 24A provides a schematic of an experimental workflow, in accordance with an exemplary embodiment of the present invention. FIG. 24B provides images of macrophages in the experiment shown in FIG. 24A. FIG. 24C provides a plot showing results of the experiment shown in FIG. 24A.

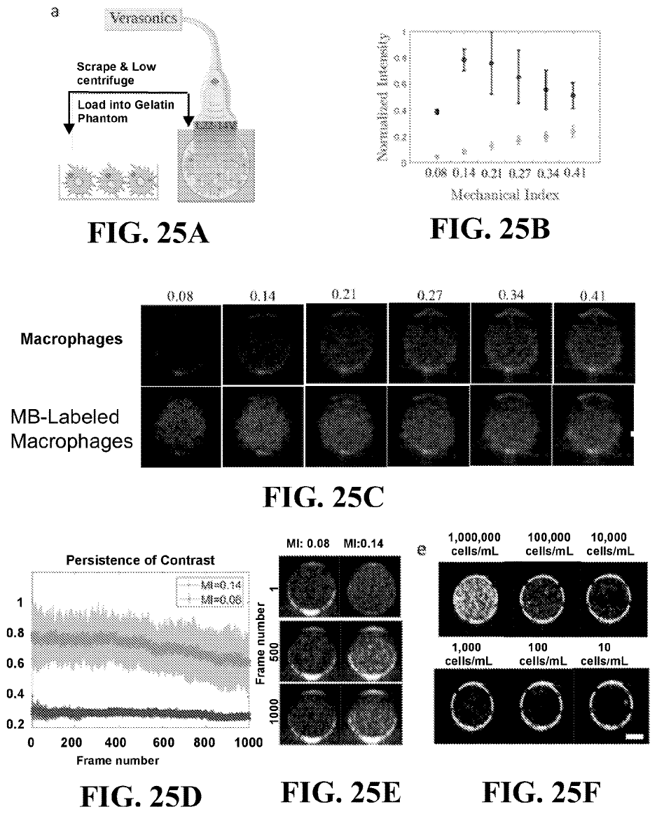

FIG. 25A provides a schematic of an experimental workflow, in accordance with an exemplary embodiment of the present invention. FIG. 25B provides a plot showing results of the experiment shown in FIG. 25A, in accordance with an exemplary embodiment of the present invention. FIG. 25C provides images of macrophages in the experiment shown in FIG. 25A. FIG. 25D provides a plot showing results of the experiment shown in FIG. 25A, in accordance with an exemplary embodiment of the present invention. FIG. 25E provides images of macrophages in the experiment shown in FIG. 25A. FIG. 25F provides images of macrophages in the experiment shown in FIG. 25A.

Various echogenic microbubbles (20a, 20b, 20c, 20d, 20e) can be employed in the methods disclosed herein. FIG. 7A shows several example microbubbles, including a fluorocarbon compound in a lipid shell and also a fluorocarbon compound in a mannose shell. Lipid microbubbles with different shell composition (mannose shell vs lipid shell) and gas core (Octafluoropropane—C3F8—labeled with ‘O’ vs Decafluorobutane—C4F10) can be employed. The FDA approved microbubble Definity is also shown.

Though the term “microbubble” is used herein, this term is not intended to include only shelled gas particles of a certain scale, micrometer or otherwise, but rather gas particles, shelled and otherwise, which are sized such that they can be contained within a cell.

The following examples further illustrate aspects of the present disclosure. However, they are in no way a limitation of the teachings or disclosure of the present disclosure as set forth herein.

### EXAMPLES

Described below are example methods for labeling macrophages with microbubble ultrasound (US) contrast agents to render them visible to ultrasound and enable high sensitivity imaging of macrophage trafficking deep into tissues. Labeling macrophages with microbubble (MB) ultrasound contrast agents (˜2 μm in size) to render them visible to ultrasound (i.e., echogenic macrophages) enables high sensitivity imaging of macrophage trafficking deep into tissues (i.e., detection of a single cell). Considering that MB labeled macrophages can act as sono-activatable point sources, super-resolution ultrasound imaging (SR-US) alleviates tradeoffs between resolution and penetration depth and effectively tracks individual macrophages (˜20 μm) and their interaction with the host tissue deep in the body. The present disclosure enables the imaging of macrophage trafficking and supports the discovery of new biomarkers for improved diagnosis and prognosis, in addition to providing a non-invasive method for monitoring macrophage-based therapy (e.g., Chimeric Antigen Receptor Macrophages-CAR-M), cell-based functional imaging for diagnosis of human disease by employing macrophages polarized towards M1/M2 phenotypes, and in situ reprograming of macrophage polarization through the application of controlled mechanical stress, which can lead to additional/different opportunities for diagnosis and treatment of human disease. While recent advances in immune cell labeling and imaging have provided new capabilities in imaging macrophage trafficking, imaging methods with high sensitivity that are also able to effectively track individual macrophages deep in the body as they interact with the host and over long timescales remains an unmet need.

Macrophages are key effector cells of the innate immune system with a wide distribution in lymphoid and non-lymphoid tissues throughout the body. Imaging of macrophage trafficking (eg. flux and density) can reveal important molecular, cellular, and functional characteristics of the host tissue (i.e., disease site) and support the discovery of new biomarkers for improved diagnosis (e.g., detection of cancer metastasis), prognosis, and treatment monitoring of human disease, including cancer and atherosclerosis. Moreover, adoptive transfer of monocyte-derived macrophages has also shown promise towards preventing the development of autoimmune type-1 diabetes and promoting antitumor immunity, among others, providing unique opportunities for therapy. Macrophages have pivotal functions in homeostasis and many physiological processes beyond innate immunity, including metabolic function, clearance of cellular debris, and tissue repair and remodeling.

The methods described herein enable tracking macrophage trafficking at clinical scales, detection of macrophages in the tissues with high sensitivity (Signal-to-Noise Ratio: SNR>5), discrimination of macrophages from background cells (i.e., high specificity), resolution of a single macrophage (i.e., spatial resolution equal to or better than 20 μm), capturing slow moving macrophages (less than 1 mm/s), which is indicative of strong interaction with the host, imaging a whole organ deep in the body (i.e., field of view—(FOV) of several cm), and high portability in order to track immune cells over long timescales (from several minutes to several hours) and capture their interaction and residence to the host. While recent advances in immune cell labeling (ex vivo) and imaging (following in vivo administration) have provided new capabilities for imaging macrophage trafficking, imaging methods with high sensitivity that are also able to effectively track individual macrophages deep in the body as they interact with the host and over long timescales remains an unmet need. See Table 1.

TABLE 1
Summary of main imaging modalities for immune cell tracking.

| Imaging | Sensitivity | Specificity | Spatial Res. | Temporal | | Field of View | |
|---|---|---|---|---|---|---|---|
| Modality | (# cells) | (cell type) | [mm] | Res. | | [mm] | Portable |
| PET | ~2 × 10³ | High | 5 | >30 | min | Whole body | Low |
| MRI | ~6 × 10⁴ | High | 0.2 | 45 | min | Whole body | Low |
| X-Ray/CT | ~1 × 10⁴ | Low | 1.4 | ~1 | min | Whole body | Low |
| Photoacoustic | ~8 × 10³ | High | 0.1 | <1 | min | 10 × 10 | High |
| Microcopy | 1 | High | 0.001 | <1 | s | 0.1 × 0.1 | Average |
| US (24 | Not | Not | Not | <1 | s | 10 × 10 | High |
| MHz) | Reported | Reported | Reported | | | | |

US imaging can potentially satisfy several of the key requirements for tracking macrophage trafficking at clinical scales. Most notably, it is a mobile, non-ionizing, and low-cost technology that can support safe and cost-effective longitudinal imaging. Unfortunately, US alone, as all other imaging modalities (Table 1), offers limited sensitivity and image contrast for discriminating specific cell populations. This is because the scattering cross section of the majority of cells, including macrophages, is essentially identical (e.g., σ_{macrophage}≈4.07×10⁻¹¹ m²; σ_{Cancer}≈8.55×10⁻¹¹ m²) and as such they produce limited to no image contrast. These limitations can potentially be overcome by labeling the macrophages with microbubbles. Microbubbles are micron-scale gas bubbles that are stabilized against dissolution by a lipid, polymer, or protein shell. As the gas acoustic impedance and, by extension, its scattering cross-section is more than thousand-fold higher than those of liquids and cells (σ_{Bubble}≈10⁻⁷ m² for a bubble with Radius=1 μm), gas Microbubbles produce sufficiently strong echoes to detect a single MB. Crucially, their highly nonlinear response provides the means to isolate their echoes from the tissue and enable imaging with high specificity. Due to their excellent image contrast, Microbubbles are increasingly used in the clinic as US contrast agents for US vascular imaging.

Moreover, ultrasound in combination with Microbubbles can alleviate tradeoffs between resolution and working distance or penetration depth that, for clinically relevant frequencies, establishes a lower bound to the imaging resolution (e.g., at 4 MHz the limit is ˜400 μm). Using Super-Resolution US (SR-US) imaging, resolution more than eightfold below the diffraction limit has been consistently reported across several studies in rodents and in human brains through intact skull. Such imaging techniques, have a theoretically achievable resolution of the order of a few micrometers for clinical US frequencies. Moreover, through the tracking of individual Microbubbles, as they flow into the vessels, microvascular flow velocities less than 1 mm/s can be identified. Together these investigations clearly underscore the abilities of US imaging to effectively detect, isolate, and track individual Microbubbles deep in the body.

FIG. 6 shows a conceptual representation of the method, and example components used therein, of the present disclosure. At a high level, first, macrophages are labeled with Microbubble Ultrasound Contrast Agents. Next, MB labeled macrophages is injected intravenously, and then the patient is imaged with conventional US or super-resolution US allowing tracking of their trafficking and form density maps of macrophage distribution and interaction to the host tissue.

Labeling macrophages with microbubble (MB) ultrasound contrast agents (approximately 1-2 μm in size) renders them visible to ultrasound (thus creating echogenic macrophages) and enables high sensitivity imaging of macrophage trafficking deep into tissues (i.e detection of a single cell). The present methods also alleviate tradeoffs between resolution and penetration depth and effectively track individual macrophages (˜20 μm) and their interaction with the host tissue deep in the body based on SR-US imaging concepts and methods. The examples disclosed herein also enable controlled mechanical stimulation of macrophages and promotion of changes in their phenotype to facilitate improved diagnosis and therapy.

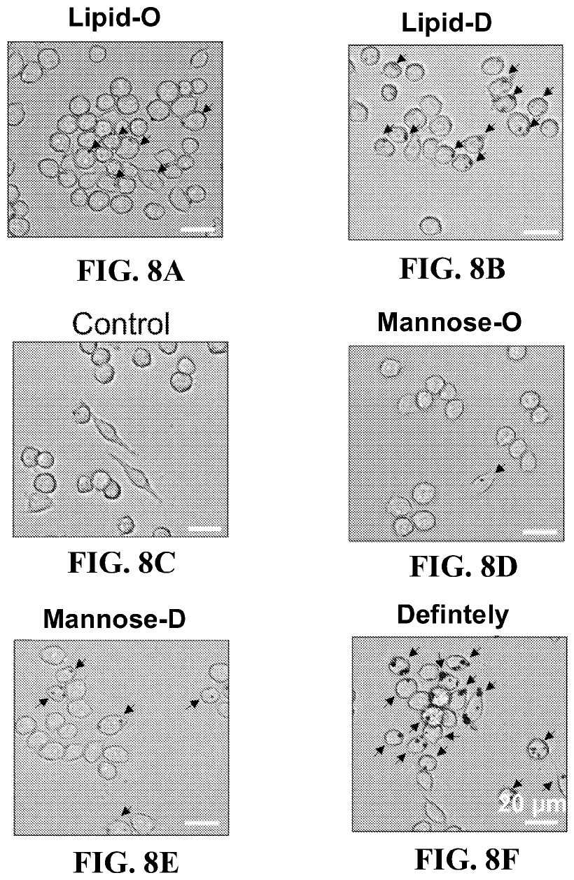

Relating to macrophage labeling with microbubbles via phagocytosis—it is an objective of the methods of the present disclosure to label macrophages with microbubbles, and this is accomplished in part by tuning the microbubble shell properties (e.g., PEG, Mannose, Fc) and charge, stiffness, and size (ranging from below 1 μm to a few micrometers). To better understand microbubble phagocytosis and its role in the creation of echogenic macrophages, the inventors developed microbubbles with different shell and gas properties (shown in FIG. 7A) and designed experimental methods and protocols to promote and study their interaction with macrophages, as shown in FIG. 7B. These preliminary in vitro investigations indicated that microbubbles with the low diffusivity gas Decafluorobutane led to higher uptake by RAW264.7 macrophages as compared to the higher diffusivity gas Octafluoropropane. FIG. 7C shows time-lapse microcopy of phagocytosed Definity microbubbles after 4 hours incubation, highlighting processes related to MB digestion and exocytosis. FIGS. 8A-8F provide images showing the labeling of the RAW264.7 macrophages with the different microbubbles employed following an incubation time of 4 hours and with black arrows indicate MB labeled macrophages. Considering that lower gas diffusion from the bubble leads to more stable bubbles, these results show that MB stability is a key MB property for effective MB uptake by macrophages.

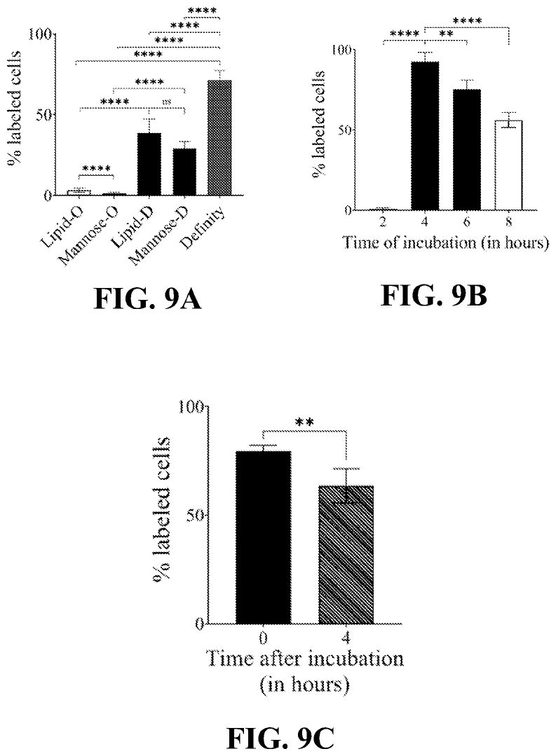

FIG. 9A shows quantification of microbubble phagocytosis using optical microscopy. The samples are washed thrice before image acquisition and quantification to remove free bubbles. FIG. 9B shows assessment of the fraction of the successfully labeled cells with Definity for different incubation times. FIG. 9C shows quantification of the persistence of phagocytosed Microbubbles, demonstrating that 60% of macrophages remain labeled for at least 4 hours. P-values are determined by one-way ANOVA, Tukey's multiple comparisons test in FIG. 9A and FIG. 9B. P values are determined by Two-tailed unpaired t-test in f (Prism 9; GraphPad). ****P<0.0001; **P<0.01; not significant (ns).

The importance of microbubble stability is further supported by results obtained using the significantly more stable and FDA-approved microbubble formulation, Definity, that demonstrated more efficient macrophage labeling as compared to the inventor's custom-built microbubbles, including mannose-based formulations. The methods herein take advantage of mannose's ability to increase particle phagocytosis. Following these observations, the inventors tested different incubation times using the Definity Microbubbles and found that 4 hours incubation produces robust labeling of the RAW264.7 macrophages (≥80% of macrophages; see FIG. 9B). These results also confirmed that the ingested Microbubbles remain within the macrophages for several hours (see FIG. 9C). Interestingly, microscopy data revealed that the population of ingested Microbubbles decays slowly through processes resembling microbubble digestion and exocytosis. Thus, the inventors effectively label macrophages with microbubbles via phagocytosis, and the majority of the phagocytosed microbubbles remain within the macrophages for several hours, thereby providing a time window that can be utilized for imaging their trafficking in vivo.

Due to their naturally high phagocytosis activity macrophages can support the development of simple, efficient, and robust labeling methods. However, phagocytosis is a time dependent process that depends on particle properties, including size and surface characteristics, among others. Moreover, phagocytosis comprises of several stages including, ingestion (uptake), digestion (degradation), and exocytosis (secretion) that particle properties play a key role in their progression and effectiveness. Beyond their ability to phagocytose a wide variety of particles, macrophages are also very responsive to environmental cues, including physical and mechanical stimuli. For example, low frequency (1 Hz), low level cyclic strain (ε=7%) can modulate their polarization towards M2 phenotype, whereas higher levels (ε=12%) to M1 phenotype. The inventors also characterize how US mediated oscillation (i.e., circumferential force) of phagocytosed microbubbles affects macrophage functionality. The inventors also characterize the uptake and persistence of microbubbles by macrophages and characterize the echogenicity, viability, and changes in the phenotype of MB-labeled macrophages in response to different US stimuli. As described in more detail below, the insights gained from these investigations allows the inventors to establish methods for MB-labeling of macrophages by harvesting phagocytosis as well as define US exposure window for retaining fully functional macrophages.

To assess the impact of microbubble properties on phagocytosis the inventors engineered microbubble formulations using DSPC and DSPE PEG2000 [1,2-distearoyl-sn-glycero-3-phosphoethanolamine-N-(polyethylene glycol)] lipids and decafluoro-butane gas under standard protocols. In addition to these microbubbles, the inventors produced mannose microbubbles by substituting standard DSPE-PEG2000 with DSPE-PEG-Mannose (50%). Microbubbles can also be functionalized to engage Fc receptors on macrophages using anti-PEG antibodies to orient the Fc domain away from the microbubbles. To assess the impact of microbubble size on phagocytosis, the produced microbubbles are separated in the following sizes: 1, 2, and 4 μm (3× sizes). To enable high throughput production monodisperse microbubble the inventors employ microfluidic based microbubble fabrication methods. The size of the collected microbubbles is confirmed before each experiment using a particle size analyzer. In total there are nine MB permutations: three different shell properties (PEG, Mannose, Fc, and IgG; 4× shell properties) with three different sizes. There is also an FDA-approved microbubbles marketed under the name Definity as control (these are polydisperse microbubbles that can be separated in size using buoyancy).

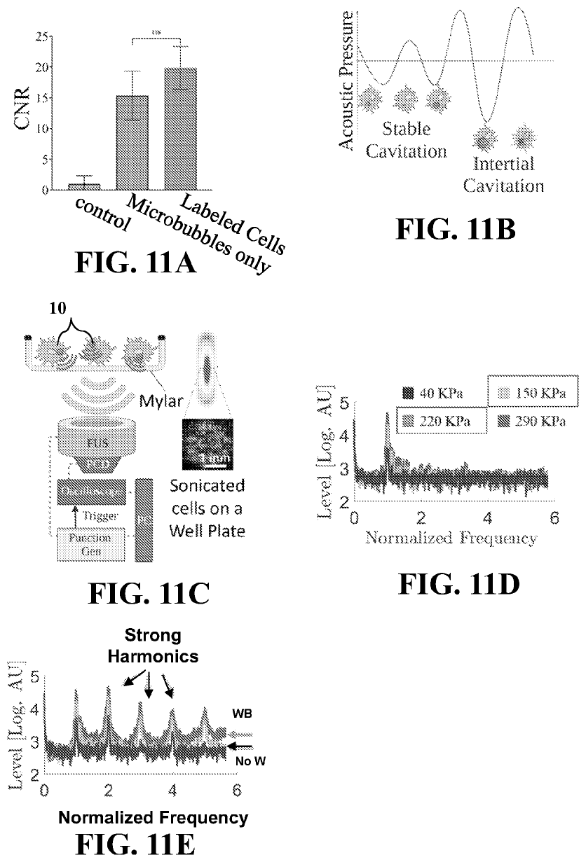

The inventors assess the uptake of these microbubbles by RAW264.7 macrophages and freshly harvested bone marrow-derived macrophages (BMDM) by co-incubating them for 4 hours using the experimental protocol shown in FIG. 7B. According to the results disclosed herein, this incubation time produces robust data. Next, the free microbubbles are removed (washed thrice) and the uptake and persistence of Microbubbles by the macrophages assessed immediately after and at 4-, 8-, 24- and 48-hours after the excess Microbubbles are removed, using optical microscopy (see FIGS. 11A-11E). Then, for the MB formulation that results in the most robust uptake (more than 75%) and persistence (more than 4 hours) by both macrophages, the incubation time and robustness in labeling the macrophages are validated by assessing their uptake over different times (0.5, 1, 2, 4, 6, 8, 12 hours) and microbubble to cell ratios (1:1, 5:1, and 10:1) (Cohort 2). The experiments in this cohort allow the inventors to identify the time to peak uptake and the conditions to attain at least one microbubble per macrophage. Note that macrophages can take up more than one microbubble with size less than 4 μm, as they are approximately 20 μm in diameter. Finally, to better understand the dynamics of MB phagocytosis and consolidate these findings, the inventors assess microbubble phagocytosis using time lapse confocal microscopy (e.g., one image per 10 seconds for 1 hour, Cohort 3). To increase the precision, accuracy, and throughput of this analysis, the inventors develop image processing routines for cell membrane and microbubble segmentation using standard functions in Matlab. Segmentation can be facilitated either by employing phase contrast imaging, which is known to enhance cell/microbubble edges, or fluorescence imaging of fluorescently labeled microbubbles (e.g., through adding a small amount of a fluorescent molecule on the microbubbles lipid) and GFP positive (RAW264.7) macrophages that provide improved delineation of the cell membrane.

Related to acoustic characterization: after identifying the microbubbles with the most efficient and robust uptake by the RAW264.7 and BMDM the inventors assess the echogenicity, viability, and changes in the phenotype of the MB labeled macrophages in response to different US exposures. By employing a 96-well plate with a mylar bottom (i.e., optically and acoustically transparent) the microbubble-labeled macrophages are sonicated at 5 different exposures (50, 100, 150, 200, 250 KPa; Cohort 4) using a 0.5 MHz FUS system that is designed to cover 75% of the area that the cells occupy in the well of the 96-well plate, while recording the microbubble acoustic emissions, as shown in FIG. 11C. Each sonication is 20 cycles long. This pulse duration is significantly larger than the ones used for imaging, thereby providing conservative estimates of the impact of microbubble oscillation on macrophages. It is also long enough to allow accurate characterization of the type of oscillation using spectral analysis of the recorded MB emissions. The microbubble emissions are recorded with a coaxially and confocally aligned passive cavitation detector (see also FIG. 11C). Spectral analysis of the recorded emissions allows the assessment of the linear and nonlinear response of the Microbubbles, as evidenced by the presence of harmonic and ultra-harmonic emissions, as well as the threshold for the onset of inertial cavitation, as evidenced by the presence of wideband emissions. At the pressure that results in strong harmonic but not wideband emissions (˜150 KPa) the inventors also assess the impact of repeated sonications on microbubble echogenicity (5×, 50×, 100× of 20 cycle bursts, Cohort 5). To ensure sufficient samples for the biological characterization of the macrophages (see below) following the sonications, each group in Cohorts 4 and 5 have at last 12 independent replicates. A summary of these experiments is provided in Table 2.

TABLE 2

| | US | | | Incubation | |
|---|---|---|---|---|---|
| Cohort | excitation | Microbubble | Macrophage | time | Evaluation |
| 1 | — | None, Build in house | RAW264.7, | 4 hours | Optical |
| | | (3x size, 4x shell), | BMDMs | | microscopy |
| | | Definity | | | |
| 2 | — | None, Build in house, | RAW264.7, | 0.5, 1, 2, | Optical |
| | | Definity | BMDMs | 4, 6, 8, 12 | microscopy |
| | | | | hours | |
| 3 | — | None, Build in house, | RAW264.7, | 4 hours | Time-lapse |
| | | Definity | BMDMs | | microscopy |
| 4 | 0, 50, 100, | None, Build in house, | RAW264.7, | 4 hours | Cell viability, |
| | 150, 200, | Definity | BMDMs | | RNASeq, ELISA |
| | 250 KPa | | | | |
| 5 | 5x, 50x, | None, Build in house, | RAW264.7, | 4 hours | Cell viability, |
| | 100x, 20- | Definity | BMDMs | | RNASeq, ELISA |
| | cycle bursts | | | | |

Biological characterization: Following the sonications first the inventors assess the macrophage viability using 4 independent biological replicates from Cohorts 4 and 5, and subsequently the inventors assess changes in their phenotype using the remaining 8 independent biological replicates (4 for RNAseq and 4 for ELISA). All groups are compared with macrophages without microbubbles. The Cell-Titer Glo luminescence-based ATP assay are used to assess cell health and viability at multiple timepoints after sonication. RNASeq is performed on Illumina sequencers in the GT Molecular Evolution Core and supported by the Applied Bioinformatics Laboratory to identify changes in mRNA transcripts induced by exposure to various US conditions in comparison to untreated and unlabeled cells. RNASeq casts a broad net and can identify changes in genes previously unknown to be related to US exposure, particularly since there is little known regarding the effect of US exposure on the macrophage transcriptome. However, changes in mRNA do not necessarily correspond to changes in protein expression or cell phenotype. The inventors also measure macrophage secretion of proteins associated with M1 and M2 polarization using ELISA. While macrophage phenotype exists on a spectrum, quantification of changes in these key proteins in response to US enables the relation of shifts toward/from M1 and M2 phenotypes. Control cells exposed to lipopolysaccharide (LPS) and IL-4 serve as comparisons for cells polarized toward M1 and M2 phenotypes, respectively. M1 associated cytokines (IL-6, IL-12, IL-23, TNF-α) and M2 associated molecules (VEGF, IL-10, PD-L1, and TGFβ) are measured 6 hours after US. Principal component analysis is used to reduce the dimensionality of the data to identify where cells with different exposures lie with respect to the M1 and M2 induced control cells.

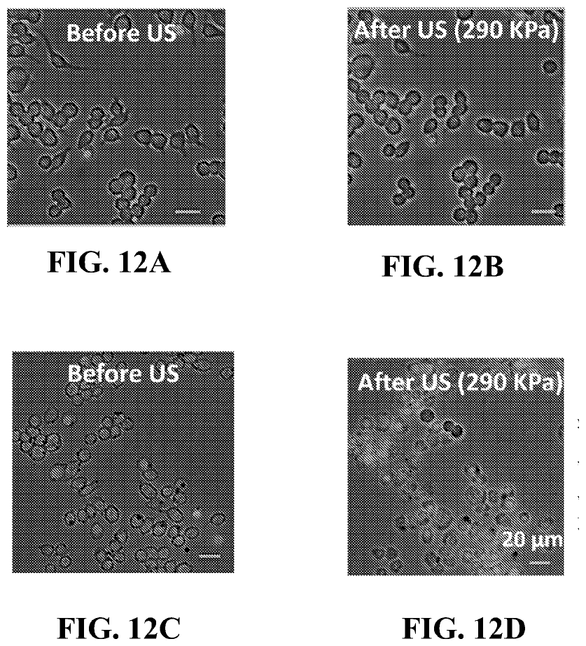

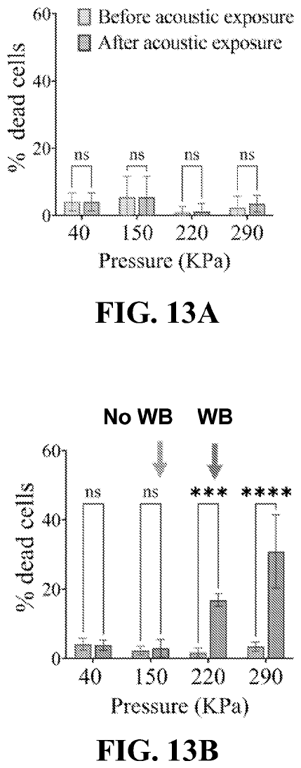

Sonicated microbubble-labeled macrophages emit strong echoes and retain high viability. To be able to track macrophage trafficking with US, the microbubble-labeled macrophages need to generate strong echoes upon ultrasonic excitation (i.e., sonication), while retaining high cell viability. To assess the echogenicity of the produced MB-labeled macrophages, the inventors built an experimental apparatus that allowed them to sonicate them on the petri-dish while recording the generated echoes during sonication. Preliminary investigations show that at 150 KPa (peak negative pressure) the microbubble-labeled macrophages emit strong harmonic emissions (i.e., evidence of highly nonlinear behavior) without adversely impacting cell viability (see FIGS. 11D-13B). Above 150 KPa, wideband emissions are clearly visible in the emissions' power spectrum, indicating the onset of microbubble collapse within the cells (inertial cavitation). At these pressures (≥220 KPa) significant increase in cell death is observed. These data provide strong evidence that the developed microbubble-labeled macrophages display high echogenicity (i.e., echogenic macrophages) within clinically relevant US exposures (i.e., pressure: 50-150 kPa; mechanical index: 0.1-0.2) while retaining high viability.

The inventors further refine labeling methods by employing microbubbles with different properties (e.g., size and surface functionalization), conduct more detailed physical and molecular characterization of the microbubble-labeled macrophages in response to different US exposures, and assess the robustness of the proposed findings using BMDMs, in addition to the RAW264.7 macrophage cell line. Despite their advantages, a key limitation of the RAW 264.7 macrophage, which is a cell line that is used in previous investigations, is that it does not possess all the properties of bone marrow-derived macrophages/monocytes, which may differentiate its trafficking patterns. Thus, freshly harvested BMDMs from mice are employed using established procedures. Moreover, past investigations either did not assess the macrophage echogenicity or are primarily focused on macrophage killing/destruction, as they are used as a drug delivery vehicle, which resulted in very narrow acoustic and/or phenotypic characterization of the MB-labeled macrophages. The results described below provide detailed characterization of the macrophage response to US excitation through validated acoustical (e.g., passive cavitation detection) and analytical assays (e.g., cell viability, ELISA, Immunofluorescence, etc.).

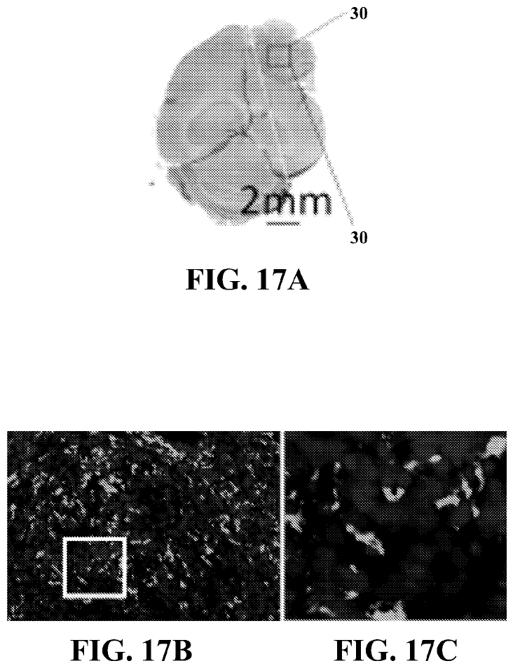

In addition to employing two different macrophage types (i.e., RAW264.7, BMDMs), the inventors employ two well-characterized cancer mice models that display high macrophage infiltration. The first is the 4T1 breast tumor model, which is a well-established tumor model that is inoculated in BALB/c mice strain, which is syngeneic to the RAW264.7 macrophage, and display high RAW264.7 macrophage infiltration. Phenotypic and functional stability of the RAW264.7 macrophages, is ensured by keeping the number of passages below ten. The second is the GL261 brain glioma tumor model that is inoculated C57BL/6J mice. This tumor model is highly infiltrated by macrophages, as shown by FIGS. 17A-17C, thereby providing a suitable model to test the proposed strategy. For the experiments with this tumor model, the BMDMs from C57BL/6J mice using established procedures. To preserve the biology and properties of the tumor microenvironment both tumors are implanted orthotopically (mammary fat pad and brain).

For experiments involving only two groups, control and experimental samples are compared by a T test (p<0.05). For experiments involving 3 or more groups, experimental conditions are compared using one-way or two-way ANOVA (p<0.05) with Tukey-Kramer adjustment for multiple comparison test.

To assess the abilities of US imaging to monitor echogenic macrophage trafficking, the inventors assess the US imaging performance of the echogenic macrophages by employing established US imaging methods (B-mode and pulse inversion) and well-defined image quality metrics (SNR and CNR) for a range of macrophage doses. These investigations is performed first in vitro, using a vessel mimicking flow through phantom, and subsequently in healthy mice (in vivo) following intravenous administration of the echogenic macrophages.

Using the same metrics, the inventors evaluate the trafficking patterns (flux and density) of echogenic macrophage in tumors with different levels of malignancy and employed well-characterized breast and brain cancer mice models that display high macrophage infiltration.

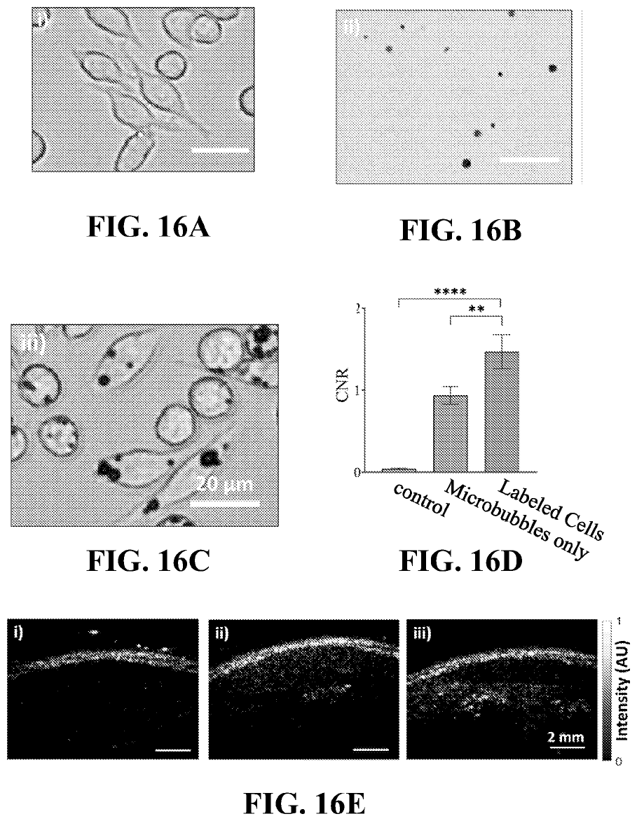

To be able to use the proposed MB-labeled macrophages, the inventors harvest them from the petri dish in large numbers, with high viability, and without negatively impacting their echogenicity. The harvested MB-labeled macrophages that are administrated in mice via tail vein injections are able to produce strong contrast in the liver of healthy mice, as shown in FIGS. 16A-16E. Interestingly, the produced Contrast-to-Noise-Ratio (CNR) is better than the one produced by an equal dose of microbubbles, presumably because microbubble-labeled macrophages had more than one bubble per cell (FIG. 16A). Considering recent evidence by others and us demonstrating that microbubble echoes are sufficiently strong to be detected through human skull, the observed CNR provides strong feasibility data for detecting single microbubble-labeled macrophage deep in tissue, including brain, under US.

The inventors determine for how long the microbubble-labeled macrophages remain in circulation, and what are the best US imaging pulse sequences for tracking their trafficking. Subsequently, the inventors analyze their trafficking patterns in diseased organ.

For the microbubble-labeled macrophages to be used in vivo, they need to be extracted from the petri dish at large quantities, while both the cells and the phagocytosed microbubbles remain intact. The cells are plated in a larger dish, such that a cell scraper can be used to extract the microbubble-labeled macrophages. Subsequently, the harvested cells are centrifugated at very low speed (100 G) for 1 minute to further separate them from free bubbles, while avoiding pellet formation and damage of the MB-labeled macrophages during centrifugation. The macrophages are collected following this protocol are shown in FIG. 16A. At the end of this procedure a fraction of the collected macrophages are tested under microscope to confirm high MB-labeling and cell viability.

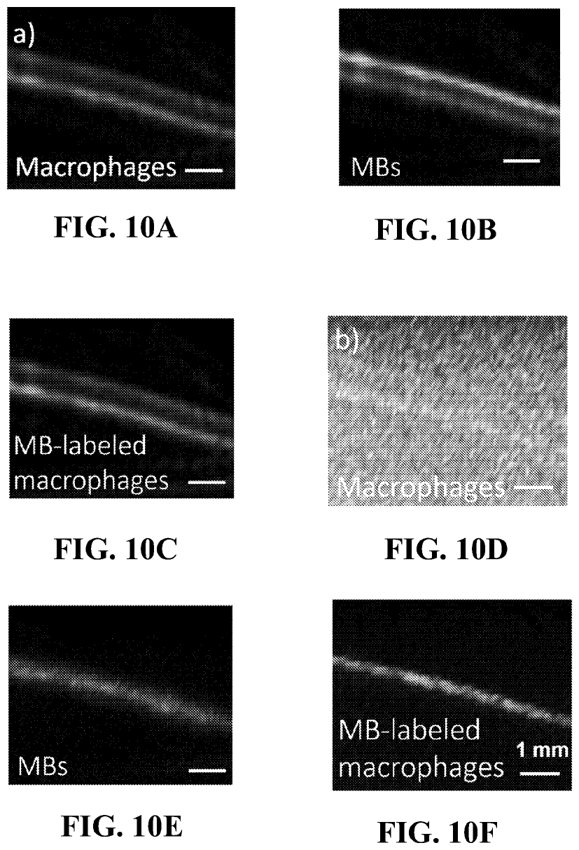

After harvesting the microbubble-labeled Macrophages (RAW264.7 and BMDMs), their imaging performance is evaluated under B-mode imaging, which is standard pulse echo technique to localize linear microbubble echoes. The image contrast of the microbubble-labeled macrophages under B-mode imaging are tested in vessel mimicking (internal diameter of 0.5 mm) flow through gelatin phantom under a flow rate of 1 ml/min. Before the experiments, the channel is sterilized (washed with alcohol, followed by saline flushing). To better distinguish the bubble signal, singular value decomposition (SVD) filtering is applied to the stack of B-mode images that contain data from the flowing microbubble-labeled macrophages. Singular values larger than 10% of the first (maximum) singular value (first two to five values) are set to 0, while the smallest singular value, which typically contains noise, are removed, as shown in FIG. 10A-10F. Although SVD filtered B-mode images produce excellent contrast a potential limitation of this approach is that it requires the macrophage motion to be different than that of the background (i.e., different spatiotemporal coherence). While this is the case in in vitro setting, in in vivo setting might result in filtering out slow moving macrophages. Hence, in addition to B-mode imaging, the inventors assess, under the same conditions, the imaging performance of the coherence-based non-linear phase inversion technique, referred to as Pulse Inversion (PI) imaging, that can explicitly image/localize nonlinear MB echoes embedded in a largely linear background. This is confirmed by assessing the CNR at flow rates below 1 ml/min.

In all experiments, the inventors employ an 18 MHz linear array transducer (L22-14D, Vermon SA, France, EU) with 1.5 mm elevation aperture, and 8 mm elevation focus. The array is controlled by the research US platform (Vantage 256, Verasonics Inc.). To establish the minimum number of macrophages that can be detected using B-mode/SVD and PI imaging the inventors employ different cell concentrations (10², 10³, 10⁴, 10⁵ labeled cells/ml) and measure the SNR and CNR using a small region of interest within the vessel and immediately adjacent to it. In these investigations, the inventors also determine the required excitation pressure by the array for effective detection, by incrementally increasing the applied voltage to the US array. Collectively, these experiments allow the inventors to refine the methods for harvesting MB-labelled macrophages (BMDM), optimize the US pulse sequencies, and assess the imaging performance of the MB-labeled macrophages through well-defined image quality metrics.

Relating to In vivo investigations (Cohort 7)—subsequently the inventors assess the US imaging performance of intravenously administered MB-labeled macrophages in the liver of healthy mice (liver is characterized by high macrophage trafficking) by employing the same imaging methods (B-mode/SVD and PI) and image quality metrics (SNR, CNR). Here, the inventors assess three different macrophage doses (10⁵, 10⁶, 10⁷ labeled cells/ml) and assess the temporal changes in the signal intensity over a 2-hour period, in addition to assessing the CNR and SNR at different time points. The macrophage flux and density are assessed by measuring the rate of change, level, and persistence of the recorded MB echoes/signal. The proposed approach and metrics to characterize macrophage trafficking in vivo is validated by assessing the macrophage accumulation, distribution, and viability in excised mice livers (i.e., after US) using immunofluorescence microscopy (see FIGS. 17A-17C).

Further experiments are carried out to evaluate the trafficking of echogenic macrophage in solid tumors. First, the inventors employ the well-established 4T1 breast mice tumor model that is characterized by high RAW264.7 macrophage infiltration. The 4T1 breast tumor cell line is inoculated on the mammary fat pad of BALB/c mice (i.e., orthotopically). Following tumor inoculation, two different doses of MB-labeled RAW264.7 macrophages are tested (e.g., 10⁶ and 10⁷ cells/ml), though this number maybe be adjusted depending on the imaging contrast produced and circulation time identified in the investigations in Cohort 7) and assessed their flux and density in well-formed tumors (˜3 mm in diameter) over a 2-hour period (Cohort 8). In these investigations, the frame rate is set between 1 and 10 frames per second. To assess abilities to track macrophage trafficking at different levels of malignancy (Cohort 9), the inventors administer the developed MB-labeled macrophages at a dose that provides robust signal in Cohort 8 at different tumor sizes (e.g. from 2 mm and until the tumor is 4 mm in diameter) and assessed their trafficking patterns (flux and density) using the US imaging method that produces the most robust CNR in Cohorts 6-8. Tumor growth and macrophage accumulation in the tumor and all vital organs (liver, spleen, lungs, and hart) is quantified with IVIS imaging using orthogonal luciferase reporters stably transduced into the RAW264.7 macrophage cells (firefly) and 4T1 cell line (renilla).

To test the robustness of these methods and the abilities of the more clinically relevant BMDM to detect tumors, the inventors employ the GL261 glioma mice tumor model that is also characterized by high macrophage infiltration, as shown in FIGS. 17A-17C. The GL261 glioma tumor cell lines is inoculated in the brain of C57BL/6J mice. In this tumor model the inventors employ the same macrophage concentration, imaging methods and metrics with Cohort 8. Likewise, to assess macrophage trafficking at brain (GL261) tumors at different levels of malignancy, the inventors follow the methods and procedures described in 4T1 tumors above (Cohort 9). To mitigate challenges associated with the high scattering from the mice skull above 10 MHz and attain high image contrast the inventors perform a craniotomy and replaced the skull with an acoustically transparent window. A summary of these experiments is provided in Table 3.

TABLE 3

| Cohort | Imaging | Macrophage | Dose [cells/ml] | Model | n/group | N |
|---|---|---|---|---|---|---|
| 6 | B-mode/ | RAW264.7, | 10², 10³, 10⁴, | In vitro | — | — |
| | SVD, PI | BMDM | 10⁵ | | | |
| 7 | B-mode/ | RAW264.7, | Ctrl*, 10⁵, 10⁶, | Healthy | 3 | 48 |
| | SVD, PI | BMDM | 10⁷ | mice | | |
| 8 | B-mode/ | RAW264.7, | Ctrl*, 10⁶, 10⁷ | 4T1, GL261 | 4 - 4T1; 6- | 120 |
| | SVD, PI | BMDM | | | GL261 | |
| 9 | B-mode | RAW264.7, | 10⁶ | 4T1, | 3 | 18 |
| | or PI | BMDM | | GL261 | | |
| | | | | (2, 3, 4 mm) | | |

| | |
|---|---|
| Total Sample Size | 186 |
| for Aim 2 | |

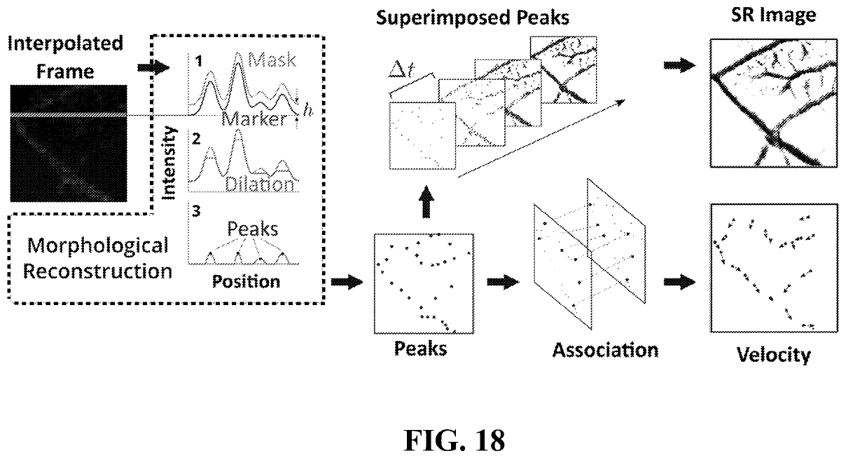

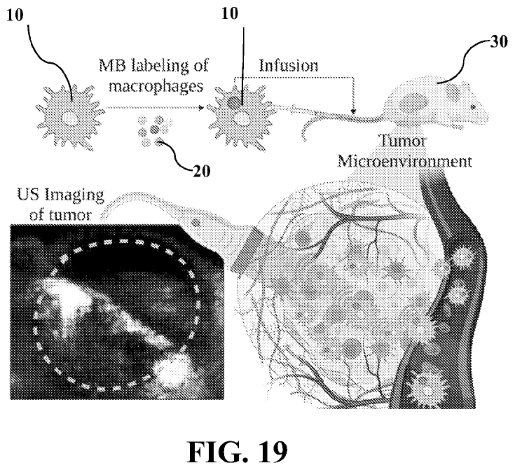

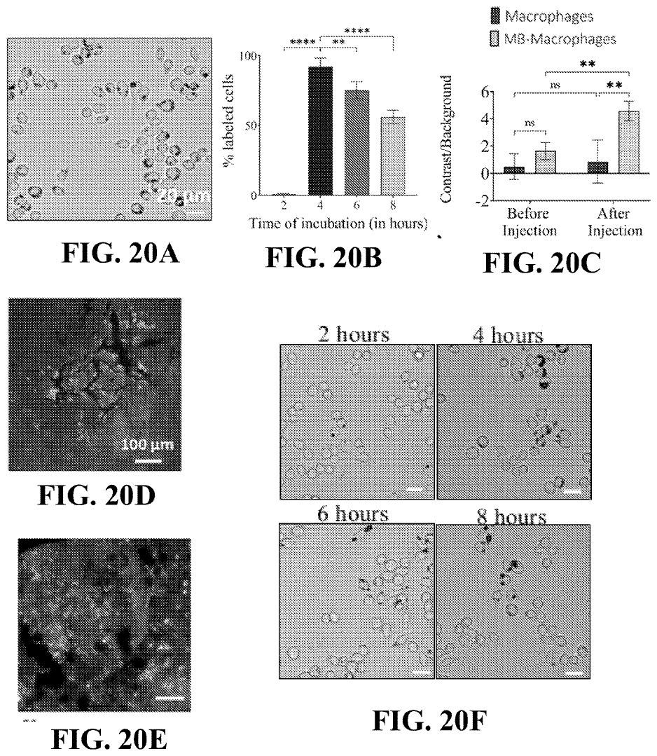

Disclosed herein is also an ultrasound framework for tracking macrophage trafficking at single cell resolution. The inventors perform investigations in vitro (Cohort 10) using a vessel mimicking tube (<100 μm inner diameter) with a bifurcation and low fluid velocities (0.01 to 10 mm/s). Although the flow rates are controlled by a high precision digital pump, the actual velocities in the channel are also be measured optically using particle image velocimetry (i.e., fluorescent beads flowing through the bifurcation is imaged under microscope). As before, US imaging is performed using B-mode/SVD filtering and PI. Because there is a need for spatiotemporal separation in SR-US, such that adjacent bubbles are separated by a distance equal to the point spread function imaging array (˜150×50 μm for the L22-14v, Vermon), the inventors employ different macrophage concentrations (e.g., 10²-10⁵ labeled cells/ml). Robust and automatic MB peak isolation routines are also extremely important in SR-US, where thousands or millions of correctly identified peaks (i.e., in thousands of frames) are needed for attaining high SNR in the final superimposed image. Thus, in addition to existing microbubble peak isolation methods based global thresholding, the ability of morphological image reconstruction to correctly identify multiple MB peaks with different intensities within an image is also assessed. For this algorithm to preform best the offset between marker and mask image (‘h’; see FIG. 18) is optimized and the spread of each peak is used to refine the search for peaks from single microbubbles. Subsequently, the super-resolved location of the isolated microbubbles is estimated using deconvolution or centroid detection methods. To estimate the flow velocity, the peaks found via morphological reconstruction is paired using a nearest-neighbor scheme (FIG. 18). That is, each peak in frame n is paired with the closest peak in frame n+1 and the velocity is estimated as follows:

$${u}_{i} = ( {r}_{i}^{n + 1} - {r}_{i}^{n} ) / Δ t ,$$

where

$${r}_{i}^{n}$$

is the position of the peak i in frame n and Δt is the time between the acquired frames. The final images are reconstructed either by projecting the detected peaks or the estimated tracks and velocities on an image grid with pixels smaller than the wavelength (10×10 μm).

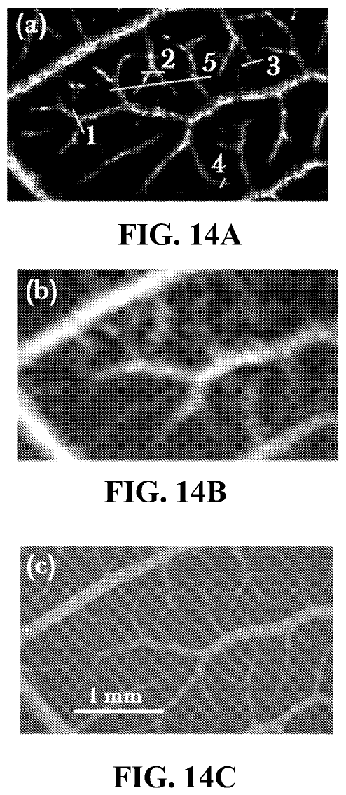

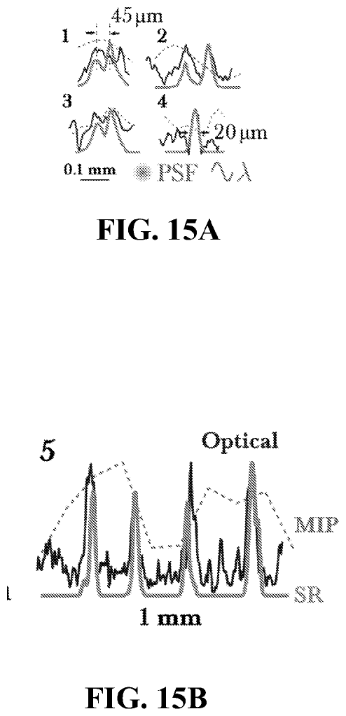

The improved spatial resolution of SR-US comes at the cost of poor temporal resolution due to the prolonged image acquisition that is required in order to spatiotemporally separate/isolate thousands of individual microbubbles. To alleviate this tradeoff, governing image quality, it is critical to identify effective methods to detect and isolate individual microbubbles over thousand frames. However, due to the stringent microbubble selection and acceptance criteria, microbubbles that are either smaller in size (i.e., below resonance size) or experience higher damping (e.g., in small capillaries), can end up below the applied detection thresholds. To improve detection of these microbubbles, which may reside at structures of interest in SR-US (e.g., micro-vessels), there is provided a computationally efficient method based on morphological image reconstruction. This method can offer more than fourfold increase in the number of peaks detected per frame (312-by-180 pixels), as compared to standard global thresholding approaches, requires on the order of 100 ms for processing, and is robust to additive electronic noise (down to 3.6-dB CNR in contrast enhanced US images). By integrating this method to a SR-US imaging framework, the inventors achieve up to a sixfold improvement in spatial resolution, when compared to contrast enhanced US (see FIGS. 14A-15B), with minimum penalty on processing time.

The inventors adapted and refined this SR-US framework to detect, isolate, and (super) localize MB-labeled macrophages and demonstrate its abilities to track macrophage trafficking at single cell resolution in the brains of healthy and tumor-bearing mice. The inventors achieved imaging of MB-labeled macrophages using B-mode imaging, singular value decomposition (SVD) filtering to a stack of B-mode images that contain data from the flowing MB-labeled macrophages, Doppler imaging, and coherence-based non-linear phase inversion technique, referred to as Pulse Inversion (PI) imaging, that can explicitly image nonlinear MB echoes embedded in a largely linear background, among others.

FIG. 16A shows optical images of non-labeled macrophages, FIG. 16B shows optical images of Microbubbles, FIG. 16C shows optical images of MB-labeled macrophages. FIG. 16D shows quantification of the image contrast of the maximum intensity projection images, demonstrating strong image contrast for the MB-labeled macrophages. P-values are determined by one-way ANOVA, Tukey's multiple comparisons test as previously described. FIG. 16E shows maximum intensity projection from 250 B-mode images of mice liver collected after the administration of i) non-labeled macrophages, ii) Microbubbles only, iii) MB-labeled macrophages. The images are obtained using an 18 MHz linear array transducer (L22-14D, Vermon) that is controlled by the Verasonics, Vantage 256 ultrasound system. The MB dose is adjusted to be equal to the dose of successfully labeled macrophages (3×10⁶ cells/ml).

Effective imaging of immune cell trafficking is ultimately dependent on these methods' ability to track individual cells as they interact with the host. SR-US imaging of MB-labeled macrophages provides unique opportunities to resolve immune cell trafficking at a single-cell resolution. While several practical aspects of SR-US (MB dose, frame rate, and MB detection along with peak extraction, isolation, tracking, and velocimetry) have been studied and advanced in the context of microvascular imaging and flow velocimetry using Microbubbles, SR-US has not been tested for tracking immune cell trafficking. Imaging of MB-labeled macrophages with SR-US may introduce additional constraints and sources of variability. Most notably, the different rheology of microbubbles (i.e., they flow in the middle of the vessel like erythrocytes) and macrophages (which roll or crawl along blood-vessel walls with velocities smaller than 1 mm/s) may challenge the limits of established SR-US implementations. For instance, USCA peak detection using frame subtraction or SVD filtering that either requires microbubble velocities larger than the underlying tissue motion or different spatiotemporal coherence might not be as effective. In addition, microbubble-labeled macrophages can have substantial variation in their scattering cross sections, depending on their location (in the vessel wall or in the tissue) and stage of phagocytosis (see FIG. 7C), which can render standard global thresholding approaches inappropriate. Hence, de novo optimization of SR-US and, where appropriate, introduction of new methods to account for the specific requirements of this application, is required for tracking individual macrophage trafficking with this imaging technique.

MB/macrophage peak detection can be performed using either using linear methods (B-mode/SVD filtering) or nonlinear methods (e.g., Pulse Inversion). This imaging can be performed for different i) MB-labeled concentrations (e.g., 10²-10⁶ labeled cells/ml), ii) frame rates (e.g., 0.01-1000 frames per second), and durations (e.g., less than 1 min to several hours).

MB/macrophage peak isolation can be performed using global thresholding methods. Morphological image reconstruction methods that can identify multiple MB peaks with different intensities within an image can also be used, among others.

To estimate the flow velocity, the peaks found via MB/macrophage peak detection and isolation (e.g., morphological reconstruction) is paired using a nearest-neighbor scheme. That is, each peak in frame a is paired with the closest peak in frame n+1 and the velocity can be estimated as follows:

$${u}_{i} = ( {r}_{i}^{n + 1} - {r}_{i}^{n} ) / Δ t ,$$

where

$${r}_{i}^{n}$$

is the position of the peak i in frame n and Δt is the time between the acquired frames. The final images can be reconstructed either by projecting the detected peaks or the estimated tracks and velocities on an image grid with pixels smaller than the wavelength (e.g., 10×10 μm). Each step (e.g., max distance to nearest neighbor) can be further refined, so the final image reflects the true velocity and resolution.

Furthermore, the present disclosure relates to methods for in situ reprograming of macrophage polarization through the application of controlled mechanical stress. Macrophages are very responsive to environmental cues, including physical and mechanical stimuli. For example, low frequency (1 Hz), low level cyclic strain (ε=7%) can modulate their polarization towards M2 phenotype, whereas higher levels (ε=12%) to M1 phenotype. The methods and tools described herein can be used to tune the macrophage polarization, for example, to promote changes in the phenotype (e.g., M1 vs M2) of the MB-labeled macrophages through controlled US stimuli of different amplitude, frequency, modulation etc. and/or to promote changes in the phenotype of the microbubble-labeled macrophages ex vivo or in vivo using MB-labeled macrophages.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A method comprising:
introducing a labeled phagocytotic cell into a patient; and
determining a location of the labeled phagocytotic cell in the patient.

2. The method of claim 1 further comprising:
labeling a phagocytotic cell with an echogenic microbubble to form the labeled phagocytotic cell; and
isolating the phagocytotic cell from the patient.

3. The method of claim 1 further comprising:
labeling a phagocytotic cell with an echogenic microbubble to form the labeled phagocytotic cell;
wherein the determining comprises ultrasound imaging the patient to determine the location of the labeled phagocytotic cell in the patient; and
wherein the labeling comprises:
placing the phagocytotic cell in a solution comprising the echogenic microbubble, the solution contained in a sealable container;
fixing the phagocytotic cell to a bottom surface of the sealable container;
sealing the sealable container;
inverting the sealable container;
incubating the phagocytotic cell for a period of time; and
washing the phagocytotic cell.

4. The method of claim 3, wherein;
the period of time is approximately 4 hours; and
incubating the phagocytotic cell comprises incubating the cell at approximately 100 degrees Fahrenheit.

5. (canceled)

6. The method of claim 3, wherein:
the echogenic microbubble is one of a plurality of echogenic microbubbles;
the phagocytotic cell is one of a plurality of phagocytotic cells;
the placing comprises placing the plurality of phagocytotic cells in a solution comprising the plurality of echogenic microbubbles, the solution contained in a sealable container;
the fixing comprises fixing the plurality of phagocytotic cells to a bottom surface of the sealable container;
the incubating comprising incubating the plurality of phagocytotic cells for a period of time; and
at least one of:
the solution comprises a ratio of phagocytotic cells to echogenic microbubbles of between approximately 1:1 and 1:50;
the solution comprises a ratio of phagocytotic cells to echogenic microbubbles of approximately 1:1;
the solution comprises a ratio of phagocytotic cells to echogenic microbubbles of approximately 1:5;
the solution comprises a ratio of phagocytotic cells to echogenic microbubbles of approximately 1:10;
the solution comprises a ratio of phagocytotic cells to echogenic microbubbles of approximately 1:20; or
the solution comprises a ratio of phagocytotic cells to echogenic microbubbles of approximately 1:50.

7.-11. (canceled)

12. The method of claim 1 further comprising:
labeling a phagocytotic cell with an echogenic microbubble to form the labeled phagocytotic cell; and
characterizing a trafficking pattern of the labeled phagocytotic cell;
wherein the determining comprises ultrasound imaging the patient to determine the location of the labeled phagocytotic cell in the patient.

13. A method comprising:
characterizing one or more trafficking patterns of labeled phagocytotic cells in a patient over a time period comprising:
detecting labeled phagocytotic cells in a patient with a Signal-to-Noise Ratio (SNR)>5;
discriminating at least a portion of the labeled phagocytotic cells from background cells;
resolving one or more single respective labeled phagocytotic cells from among the labeled phagocytotic cells using a spatial resolution equal to or better than 20 μm;
determining a respective velocity of at least a portion of the respective resolved single labeled phagocytotic cells, wherein a low velocity correlates with a strength of an interaction of the respective resolved single labeled phagocytotic cell with a target anatomy; and
imaging the target anatomy with a field of view (FOV) on the order of one or more centimeters.

14. A method comprising:
labeling phagocytotic cells with echogenic microbubbles;
introducing the labeled phagocytotic cells into a patient;
ultrasound imaging the patient to determine a characterization of one or more trafficking patterns of the labeled phagocytotic cells;
wherein the characterization comprises one or more of:
determining accumulation of the labeled phagocytotic cells;
determining a flux of the labeled phagocytotic cells; or
determining a distribution of the labeled phagocytotic cells.

15.-16. (canceled)

17. The method of claim 14, wherein the phagocytotic cells comprise macrophages.

18. The method of claim 14, wherein the phagocytotic cells comprise bone marrow-derived macrophages.

19. The method of claim 14, wherein the echogenic microbubbles comprise a fluorocarbon compound in a lipid shell.

20. The method of claim 19, wherein the fluorocarbon compound comprises one or more of octoflouropropane and perflourobutane.

21. The method of claim 14, wherein the echogenic microbubbles comprise a fluorocarbon compound in a mannose shell.

22. The method of claim 21, wherein the fluorocarbon compound comprises one or more of octoflouropropane and perflourobutane.

23. The method of claim 13 further comprising:
performing cell engineering on phagocytotic cells;
labeling the phagocytotic cells with an echogenic microbubble to form the labeled phagocytotic cells;
introducing the labeled phagocytotic cells into the patient; and
ultrasound imaging the patient to determine one or more locations of the labeled phagocytotic cells in the patient.

24. The method of claim 23 further comprising:
isolating at least a portion of the labeled phagocytotic cells from the patient.

25.-38. (canceled)

39. The method of claim 23, wherein:
the phagocytotic cells comprises a macrophages; and
the echogenic microbubbles comprises a fluorocarbon compound in a shell selected from group consisting of a lipid shell and a mannose shell.

40. The method of claim 39, wherein the macrophages comprise bone marrow-derived macrophages.

41. (canceled)

42. The method of claim 39, wherein the fluorocarbon compound comprises one or more of octoflouropropane and perflourobutane.

43.-44. (canceled)

45. The method of claim 23, wherein the performing comprises changing a phenotype of the phagocytotic cells.

46. The method of claim 23 further comprising:
determining the target anatomy based on the one or more trafficking patterns; and
administering a therapeutic to the target anatomy.
